(19) United States
(12) Patent Application Publication
(10) Pub. No.: US 2011/0208200 A1
(43) Pub. Date: Aug. 25, 2011

(54) VETERINARY PRECISION FIXATION DEVICE AND METHOD OF USING THE SAME

(76) Inventor: Gary Keffer, Stilwell, KS (US)

(21) Appl. No.: 13/101,576

(22) Filed: May 5, 2011

Related U.S. Application Data
(63) Continuation-in-part of application No. 11/464,722, filed on Aug. 15, 2006, now abandoned.

(51) Int. Cl. A61B 17/56

(52) U.S. Cl. 606/87

(57) ABSTRACT

A veterinary precision fixation device for use in tibial plateau leveling osteotomy procedures includes a jig assembly having a center jig member having a first and a second end, a first jig leg pivotally coupled to the first end, and a second jig leg pivotally coupled to the second end. The device also includes a saw positioning assembly including a positioning arm and a guide leg, the positioning assembly being operably connected to the jig assembly. The device further includes a saw guide assembly having a saw fixation member slidably coupled to at least one slide bar wherein the slide bar is coupled to the guide leg of the saw positioning assembly. A method for using the precision fixation device in a tibial plateau leveling osteotomy is also provided for making a substantially perpendicular cut of the tibia.

### CROSS REFERENCE TO RELATED APPLICATIONS

This application is a continuation-in-part of and claims priority to U.S. patent application Ser. No. 11/464,722, which was filed on Aug. 15, 2006, which is hereby incorporated by reference herein to the extent permitted by law.

### BACKGROUND OF THE INVENTION

The most common condition causing either acute or chronic hind leg lameness in dogs is injury to the anterior cruciate ligament, and subsequent instability in the knee joint. This results in severe cartilage erosion and degenerative joint disease. This arthritic condition is progressive, and without surgery leads to permanent weight-bearing lameness. Once a cruciate ligament injury or tear occurs, the anatomic stability of the joint is permanently changed.

The canine or feline stifle (knee) joint shares basic similarity to the human knee joint. The lower end of the femur rests on two doughnut-shaped cartilages (menisci) that sit on top of the tibia (tibial plateau). Two internal ligaments, the anterior and posterior cruciate ligaments, work to reduce shearing forces. The knee cap (patella) attaches to the tibia by the patellar ligament and acts to extend the stifle. The medial and lateral collateral ligaments stabilize the stifle joint side-to-side. Other internal ligaments stabilize the meniscal pads during flexing and extension.

In both the canine and feline patient, the femur rest on a tibial plateau that slopes to the back of the stifle. This slope varies by the animal's breed, size, and by individual and can vary from 5 to 40 degrees. This posterior slope results in a constant backward or posterior sliding motion (thrust) of the lower femur on the menisci and tibial plateau. This posterior thrust by the femur is countered by the anterior cruciate ligament (ACL) inside the stifle joint. It is common for the ACL to partially or completely tear as a result of normal shearing forces within the joint brought on by aging, wear and tear, and athletic activity. The incidence of injury in animals is far more common than in humans, and the surgical repair is complex. Abnormal shearing forces tear the meniscal cartilages and ulcerate the femoral condyles and truchlear groove, as the patella moves upward with normal extension of the leg.

A bone-cutting procedure that decreases the weight-bearing slope of the tibia to nearly 5° has been described by Barclay Slocum in U.S. Pat. No. 4,677,973 entitled “Proximal, tibial osteotomy for leveling a tibial plateau.” The procedure described therein hinges on accurately assessing the individual patient's joint pathology, surgically changing the slope without changing other forces within the joint, and assessing, and then correcting, any meniscal damage. The surgical procedure, as disclosed by the '973 patent and its progeny, encompasses a specific surgical approach to the stifle joint and proximal tibia and then the attachment of a jig to the tibia. This is accomplished primarily by visualizing certain landmarks and making a freehand, curved, but ideally perpendicular, cut through the tibial plateau with a vibrating oscillating saw. The procedure described in the '973 patent is open to severe error because the surgeon must simultaneously visually hold the saw in three dimensions as it cuts freehand through the tibia. This procedure maximizes error in positioning the saw and therefore the cut itself.

Accordingly, there is a need in the art for a device that helps to control the freehand cut made by the oscillating curved saw blade. There is also a need in the art for a jig that is less subjectively applied to the tibia thereby reducing human error in placement.

### SUMMARY OF THE INVENTION

This invention relates generally to a veterinary surgical device and a method of using a veterinary surgical device. In particular, the device as placed by my (laser) protocol will accurately cut the tibial plateau perpendicular to the anterior-posterior plane and distal-proximal (linear) plane every time, allowing for perfect rotation and realignment of the plateau and thereby minimizing surgeon induced errors that previously occurred frequently as a result of cutting the plateau freehand or without a guide. In particular, the present invention relates to a precision fixation device for use in tibial plateau leveling osteotomies that reduces surgical error, assists in providing a concise bone cut, and results in minimal bone and soft tissue destruction during surgery. The veterinary precision fixation device of the present invention generally includes a jig assembly having a center jig member having a first and second end, a first jig leg pivotally coupled to the first end of the center jig member, a second jig leg pivotally coupled to the second end of the center jig member, a saw positioning assembly including a positioning arm having a first end and a second end, the positioning arm being operably connected to the center jig member, and a guide leg pivotally coupled to the first end of the positioning arm. The device also includes a saw guide assembly having a saw fixation member slidably coupled to at least one slide bar wherein the slide bar is coupled to the guide leg proximate the first end of the positioning arm wherein the position of the saw guide can be set by sliding or rotating the positioning arm and/or guide leg to slide and/or rotate the saw to be positioned at a desired cutting position.

### BRIEF DESCRIPTION OF THE SEVERAL VIEWS OF THE DRAWING

In the accompanying drawings that form a part of the specification and that are to be read in conjunction therewith and in which like reference numerals are used to indicate like parts in the various views:

FIG. 1 is a perspective view of the precision fixation device in accordance with one embodiment of the present invention;

FIG. 2 is a perspective exploded view of the precision fixation device in accordance with one embodiment of the present invention;

FIG. 3 is a perspective view of the precision fixation device shown in conjunction with an oscillating saw and an animal's tibia in phantom in accordance with one embodiment of the present invention;

FIG. 4 is a perspective view of a slide bar in accordance with one embodiment of the present invention;

FIG. 5 is a perspective view of the center jig member in accordance with one embodiment of the present invention;

FIG. 6 is a perspective view of the saw guide member in accordance with one embodiment of the present invention;

FIG. 7 is a perspective view of one of the jig legs in accordance with one embodiment of the present invention;

FIG. 8 is a perspective view of an adjustment member in accordance with one embodiment of the present invention;

FIG. 9 is a perspective view of the guide leg in accordance with one embodiment of the present invention; and

FIG. 10 is a perspective view of the positioning arm in accordance with one embodiment of the present invention.

### DETAILED DESCRIPTION OF THE INVENTION

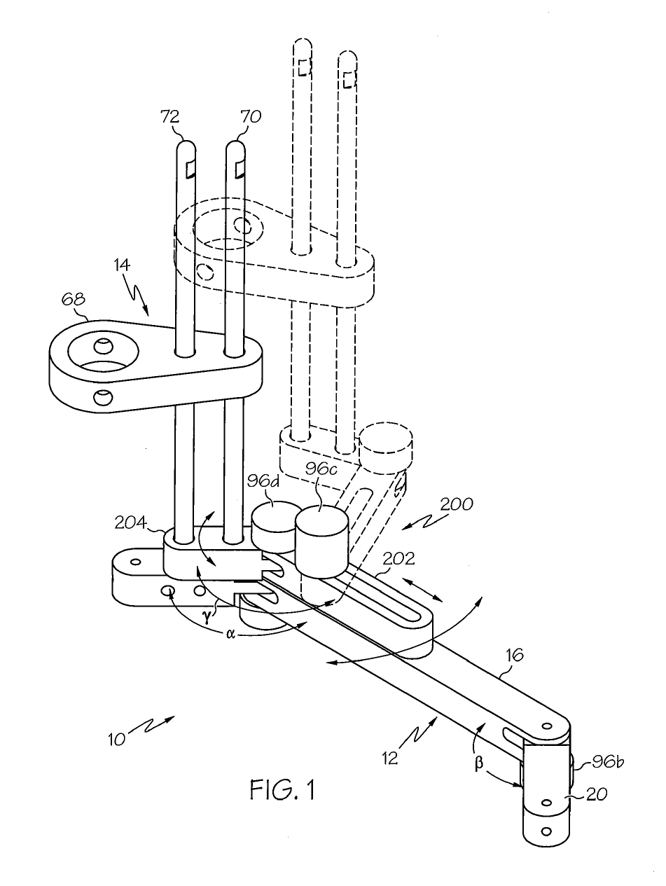

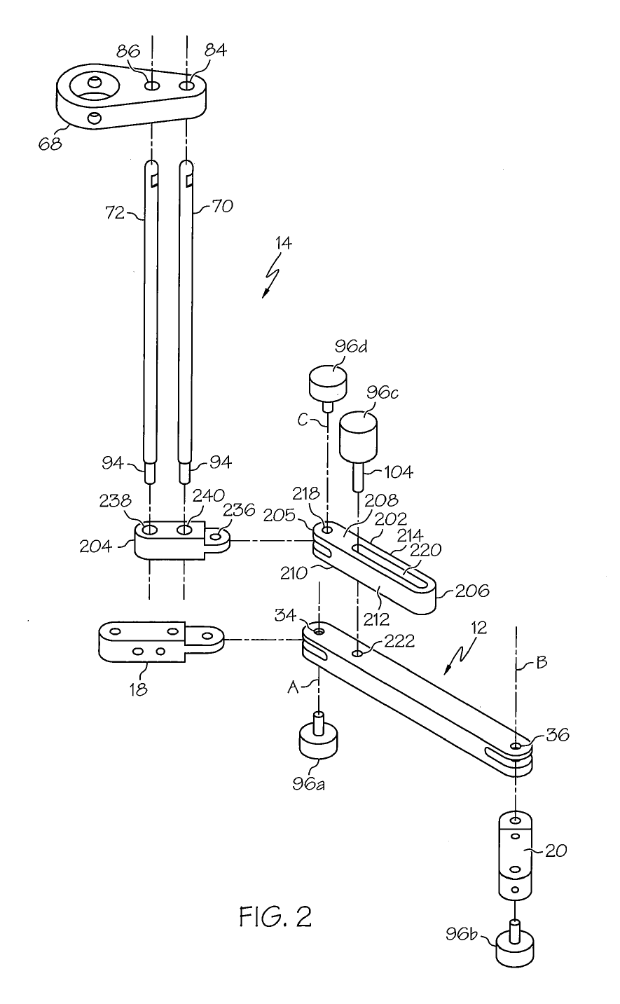

As shown in FIGS. 1-3, the precision fixation device 10 of the present invention includes a jig assembly 12 and a saw guide assembly 14. When used, device 10 of the present invention allows the user to control the accuracy of getting a substantially perpendicular cut along the axis of the tibia. In particular, the device as placed by my (laser) protocol will accurately cut the tibial plateau perpendicular to the anterior-posterior plane and distal-proximal (linear) plane every time, allowing for perfect rotation and realignment of the plateau and thereby minimizing surgeon induced errors that previously occurred frequently as a result of cutting the plateau freehand or without a guide. A method for using the precision fixation device in a tibial plateau leveling osteotomy is also provided for making a substantially perpendicular cut of the tibia.Jig assembly 12 is preferably formed from stainless steel, plastic, aluminum or any other material suitable for use in veterinary surgery. Jig assembly 12 includes a center jig member 16, a first jig leg 18, a second jig leg 20, and a saw positioning assembly 200. Center jig member 16, as shown in FIG. 5, is preferably from about 3.0-6.0 inches in length, from about 0.5-0.7 inches wide, and from about 0.3-0.6 inches high. However, it will be appreciated by one skilled in the art that the dimensions of center jig member 16 may be altered to suit the size of the animal being operated upon or to meet any other desired application. Center jig member 16 includes a generally planar or flat top surface 22, an opposing generally planar bottom surface 24, a generally planar inner surface 26, an opposing generally planar outer surface 28, a generally rounded first end 30, and an opposing generally rounded second end 32. Each of first and second ends 30 and 32 include a first and second aperture 34 and 36, respectively, extending through center jig member 16 from top surface 22 to bottom surface 24. Further, center jig member 16 includes a positioning arm attachment aperture 222 extending through center jig member 16 from top surface 22 to bottom surface 24. In one embodiment shown in FIGS. 2 and 5, aperture 222 is located between apertures 34 and 36. In one embodiment as shown in FIG. 5, aperture 222 is closer to first end 30 than second end 32. In general, the position of aperture 222 can be anywhere on center jig member 16 that allows positioning arm 202 to be adjusted to locate saw guide assembly 14 in a desired position.

Apertures 34, 36 and 222 preferably have a diameter in the range from about 0.15-0.2 inches and are configured to receive a screw 104 of an adjustment member 96 described hereinbelow. In one embodiment, apertures 34, 36 and 222 may have a threaded interior surface configured for receiving screw 104 or other threaded coupling mechanism therethrough. However, it is within the scope of this invention that the interior surface of apertures 34, 36, and 222 may be smooth or otherwise configured to receive any suitable coupling mechanism including, but not limited to, bolts, clamping mechanisms, pins, springs, or any other coupling mechanism known in the art.

As seen in FIG. 5, each of first and second ends 30 and 32 also include first and second cutout portions 38 and 40, respectively, each having a height in the range from about 0.16 to 0.2 inches and extend inwardly from each of ends 30 and 32 about 0.3-0.7 inches toward the center of center jig member 16. Cutout portions 38 and 40 are configured to receive a second end 52 of each of first and second jig legs 18 and 20 and are preferably disposed about 0.18-0.24 inches below top surface 22 and about 0.9-0.20 inches above bottom surface 24. First and second apertures 34 and 36 are preferably spaced from about 0.2-0.4 inches from first and second ends 30 and 32, respectively, with a distance between first and second apertures 34 and 36 of from about 2.0-6.0 inches such that apertures 34 and 36 are interrupted by cutout portions 38 and 40.

First and second jig legs 18 and 20, shown in FIG. 7, are each preferably from about 1.0-4.0 inches long, from about 0.3-0.7 inches wide, and include a generally planar top surface 42, an opposed generally planer bottom surface 44, a generally planar inner surface 46, an opposed generally planar outer surface 48, a generally rounded first end 50 and a generally rounded second end 52. The height of jig legs 18 and 20 at first end 50 is preferably from about 0.3-0.7 inches and tapers or reduces approximately halfway along length to a height of from about 0.16 to 0.2 inches at second end 52. It will be appreciated that second end 52 is configured to be inserted into cutout portions 38 and 40. Jig legs 18 and 20 each include an adjustment member aperture 54 extending through the top surface and the bottom surface of the reduced height end portion proximate second end 52. The position of adjustment member aperture 54 is configured so that when second end 52 is inserted into cutout portions 38 and 40 of center jig member 16, adjustment member aperture 54 lines up with first and second apertures 34 and 36 in center jig member 16 and adjustment members 96a and b described hereinbelow are inserted through center jig member 16 and second end 52 of jig legs 18 and 20 thereby enabling adjustable pivotal movement of jig leg 18 or 20. Jig leg 18 may be rotated relative to center jig member 16 about axis A (shown in FIG. 2) wherein the angle of rotation α as shown in FIG. 1 may range from 30-330 degrees. Jig leg 20 may be rotated relative to center jig member 16 about axis B (shown in FIG. 2) wherein the angle of rotation as shown in FIG. 1 may range from 30-330 degrees.

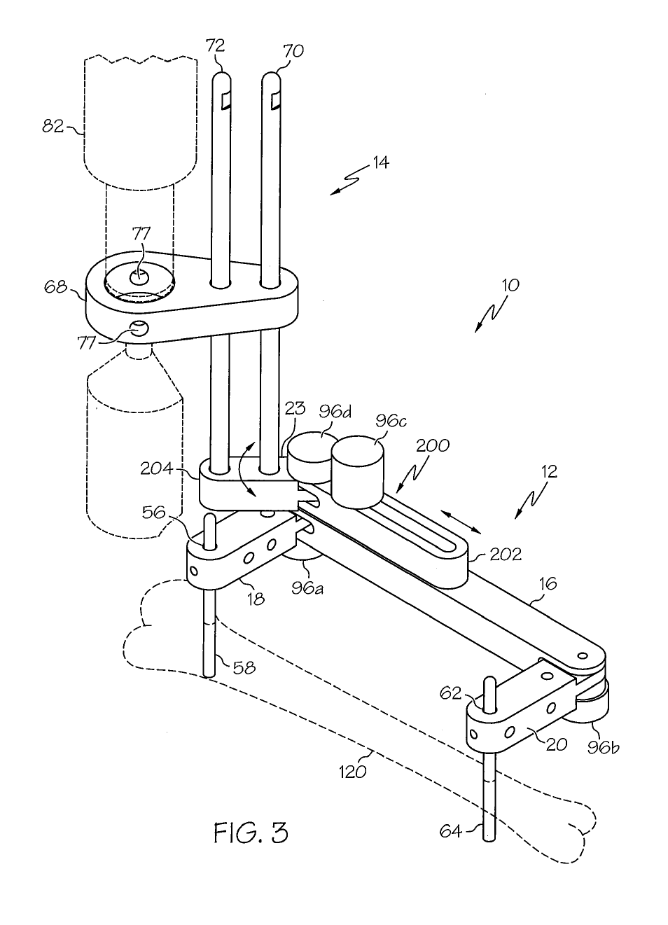

An upper tibial positioning aperture 56 extending through first end 50 of first jig leg 18 from top surface 42 to bottom surface 44 is configured to receive an upper tibial pilot pin 58 therethrough as shown in FIG. 3. A lower tibial positioning aperture 62 extending through first end 50 of second jig leg 20 from top surface 42 to bottom surface 44 is configured to receive a lower tibial pilot pin 64 therethrough as shown in FIG. 3. Second jig leg 20 may be identical to first jig leg 18 for interchangeably as shown in FIG. 7.

As shown in FIGS. 1 and 3, saw positioning assembly 200 includes a positioning arm 202 and a guide leg 204. As shown in FIGS. 1-3 and 10, positioning arm 202 is preferably from about 1.5-4.0 inches in length, from about 0.5-0.7 inches wide, and from about 0.3-0.6 inches high. However, it will be appreciated by one skilled in the art that the dimensions of positioning arm 202 may be altered to suit the size of the animal being operated upon or to meet any other desired application. As shown in FIG. 1, positioning arm 202 is generally pivotally coupled to top face 22 of central jig member 16. As shown in FIG. 10, and labeled in view of its position as shown in FIG. 1, positioning arm 202 includes a generally rounded first end 205, an opposing generally rounded second end 206, a generally planar or flat top surface 208, an opposing generally planar bottom surface 210, a generally planar first side surface 212, and an opposing generally planar second side surface 214. Positioning arm 202 also includes a cutout portion 216 having a height of from about 0.16 to 0.2 inches and extending inwardly from first end 205 about 0.3-0.7 inches toward the center of positioning arm 202. Cutout portion 216 is configured to receive a second end 234 of guide leg 204 and is preferably disposed about 0.08-0.25 inches below top surface 208 and about 0.8-0.25 inches above bottom surface 210. Positioning arm 202 also includes an aperture 218 extending through positioning arm 202 from top surface 208 to bottom surface 210. Aperture 218 is preferably spaced from about 0.2-0.4 inches from first end 205 and is interrupted by cutout portion 216.

As shown in FIG. 10, positioning arm 202 further includes slot 220. Slot 220 extends through positioning arm 202 from top surface 208 to bottom surface 210 and preferably has a length from about 0.5-3.5 inches and a width from about 0.1-0.5 inches. Each end of slot 220 may be rounded as shown in FIG. 10. Slot 220 is generally configured to have a width slightly greater than the diameter of screw 104 of adjustment member 96c as shown in FIG. 2. In one embodiment, shown in FIG. 2, screw 104 of an adjustment member 96c extends through slot 220 and is received by aperture 222 of center jig member 16. In general, at least a portion of bottom surface 210 of positioning arm 202 bears upon top surface 22 of center jig member 16. As shown in FIG. 1, the linear and radial position of position arm 202 relative to center jig member 16 can be adjusted by sliding positioning arm 202 along the length of the slot or rotating positioning arm 202 from 0-360 degrees about screw 104 of adjustment member 96c relative to center jig member 16. In one embodiment shown in FIGS. 1-3, adjustment member 96c can be rotated within the threaded aperture 222 to tighten and temporarily fix the position and radial orientation of positioning arm 202 relative to center jig member 16. In another embodiment (not shown), a rod or threaded rod is machined, glued, welded, or otherwise coupled to top surface 22 of center jig member 16 and extends outwardly wherein a nut, clamp, or other mechanism to compress bottom surface 210 of positioning arm 202 against top surface 22 of center jig member 16 to use compression and created friction to temporarily fix the position of positioning arm 202 relative to center jig member 16.

As shown in FIG. 9, guide leg 204 is preferably from about 1.0-4.0 inches long, from about 0.3-0.7 inches wide, and includes a generally planar top surface 224, an opposed generally planer bottom surface 226, a generally planar first side surface 228, an opposed generally planar second side surface 230, a generally rounded first end 232 and a generally rounded second end 234. The height of guide leg 204 at first end 232 is preferably from about 0.3-0.7 inches and tapers or reduces approximately halfway along length to a height of from about 0.16 to 0.2 inches at second end 234. It will be appreciated that second end 234 is configured to be inserted into cutout portion 216 of positioning arm 202. Guide leg 204 includes an adjustment member aperture 236 extending from top surface 224 to bottom surface 226 and proximate second end 234. As shown in FIG. 2, when second end 234 is inserted into cutout portion 216 of positioning arm 202, adjustment member aperture 236 lines up with aperture 218 in positioning arm 202 such that adjustment member 96d described hereinbelow may be inserted through positioning arm 202 and first end 205 thereby enabling adjustable pivotal movement of guide leg 204. Guide leg 204 may be rotated relative to positioning arm 202 about axis C (shown in FIG. 2) wherein the angle of rotation γ as shown in FIG. 1 may range from 30-330 degrees.

Guide leg 204 also includes a first slide bar aperture 238 and a second slide bar aperture 240, slide bar apertures 238 and 240 extending generally through the center of guide leg 204 from top surface 224 to bottom surface 226 are configured to threadably receive a first slide bar 70 and a second slide bar 72 therein or therethrough. In one embodiment, slide bar apertures 238 and 240 may have a threaded interior surface configured for receiving threaded end 90 of slide bars 70, 72 as shown in FIGS. 1-4. However, it is within the scope of this invention that the interior surface of slide bar apertures 238 and 240 may be smooth or otherwise configured to receive any suitable coupling mechanism for coupling first slide bar 70 or second slide bar 72 to guide leg 204.

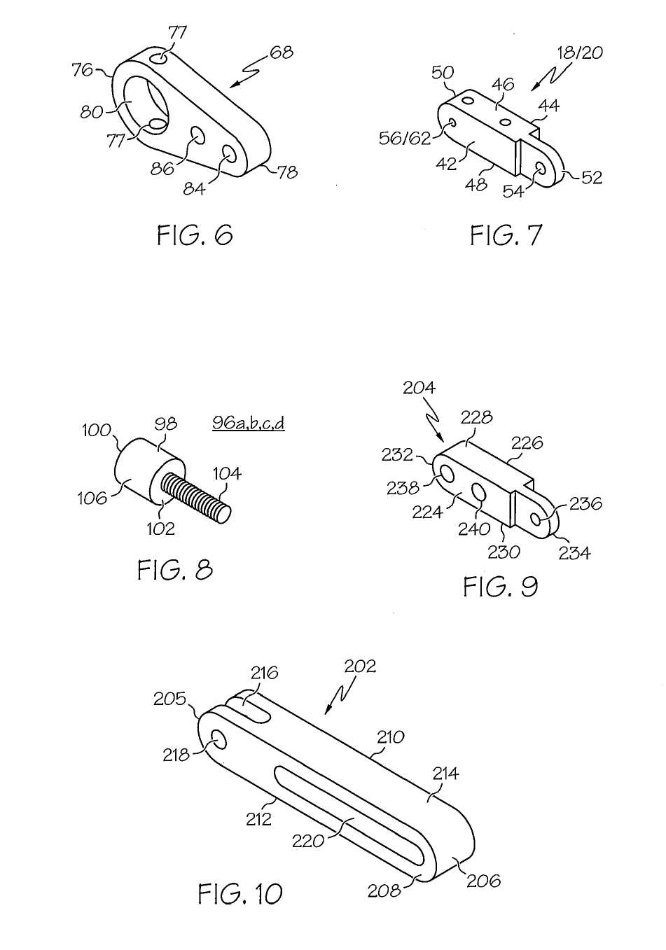

As seen in FIGS. 1-3, saw guide assembly 14 includes saw fixation member 68, a first slide bar 70, a second slide bar 72. Saw guide assembly 14 is preferably formed from stainless steel, plastic, or any other material suitable for use in veterinary surgery. Saw fixation member 68 may be any shape having rounded edges for safety purposes. In one embodiment as shown in FIG. 6, saw fixation member 68 has a roughly triangular shape with rounded edges such that a first end 76 has a larger width than a second end 78. For example, first end 76 is preferably from about 1.2 to 1.9 inches wide at its widest point whereas second end 78 is preferably from about 0.4-1.0 inches wide at its widest point. First end 76 includes a saw positioning aperture 80 extending therethrough configured for receiving and supporting a vibrating oscillating saw 82 therein and therethrough as shown in phantom in FIG. 3. First end 76 also includes opposing set screw apertures 77 for receiving set screws (not shown) for tightening and securing saw 82 within saw positioning aperture 80. Second end 78 includes a first slide bar aperture 84 extending therethrough configured for receiving first slide bar 70 therethrough. Saw fixation member 68 also includes a second slide bar aperture 86 extending therethrough configured for receiving second slide bar 72 therethrough and located between saw positioning aperture 80 and first slide bar aperture 84 as shown in FIGS. 2 and 6.

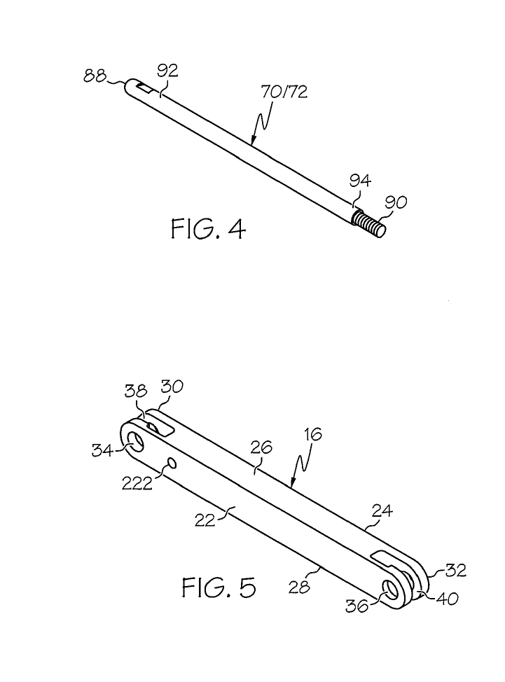

First and second slide bars 70 and 72 as shown in FIG. 4 are generally cylindrical having a rounded first end 88 and a threaded second end 90 for threadably engaging slide bar apertures 238 and 240 in guide leg 204. In another embodiment, second end 94 of slide bars 70, 72 may be welded, soldered, or simply glued to top surface 224 of guide leg 204 or within slide bar apertures 238 and 240 respectively.

As seen in FIG. 8, adjustment member 96a,b,c,d includes a generally O-shaped shoulder 98 having a top surface 100 and a bottom surface 102. Bottom surface 102 of adjustment members 96a,b,c,d includes a screw 104 extending outwardly therefrom and configured to threadably couple first and second jig legs 18, 20 to center jig member 16, positioning arm 202 to center jig member 26, and/or guide leg 204 to positioning arm 202. In the preferred embodiment, screw 104 has a length sufficient to extend through the two members being connected and create sufficient tightening force to temporarily fix one member relative to the other.

The method of the present invention minimizing positioning errors that are typical in other methods for this type of operation. The device of the present invention allows a surgeon to control the accuracy of having a perpendicular cut because the patients tibia is always substantially perpendicular to the saw. In the method of the present invention the patient is positioned in lateral recumbency and the surgery leg placed on the table, in a substantially perfect horizontal plane with stifle and tibiotarsal joints partially flexed. After the patient is placed in lateral recumbency, a vacuum positioner bag is placed beneath the surgical leg. A horizontal laser beam is used to position the distal leg, from just above the patella, so that the horizontal beam of the laser lays center of the patellar ligament, center of the proximal to distal tibia, and to the center of the tarsus. Another laser beam is used to position the leg from anterior to posterior of the leg. The vacuum positioner bag is deflated when the leg is parallel to both horizontal beams. The leg is held in passive horizontal position for the entire surgical procedure. When used in this surgery, the device of the present invention holds and guides the saw at the perfect position to ensure a correct cut.

A ⅛″ threaded pin is inserted through a small stab wound approximately 5 mm distal to the second 25 gauge needle and midway between the posterior edge of the medial collateral ligament and the third 25 gauge needle. The pin is started to make a small pilot hole and then removed.

This procedure may be enhanced through the alignment and positioning of elements for this procedure being performed using a laser positioning or alignment systems or devices as known in the art, though use of a laser guide shall not limit the scope of the present invention. In one embodiment, prior to drilling the proximal pin, the laser horizontal beam is used to confirm that the lower leg is in a substantially perfect horizontal plane. If the leg is positioned correctly, the laser beam bisects the patella, the patellar ligament, the tibia, and the metatarsus. In general, if the leg is positioned correctly prior to surgery on the vacuum positioner bag and, if the leg has not been repositioned, then no adjustments in positioning of the leg should be necessary.

Preplaced 25 gauge needles identify the posterior edge of the proximal tibia 120, and the posterior edge of the femorotibial joint. A 25 gauge needle is placed just caudal to the medial collateral ligament in the joint to identify the tibial plateau. A second 25 gauge needle is “walked” off the posterior-proximal edge of tibia 120. The tip of the proximal pin is placed 2-5 mm caudal to the medial collateral ligament, and 5-8 mm below the tibial plateau. In general the pin will be placed caudal of the medial collateral ligament, ⅔ of the distance between the medial collateral ligament and the posterior edge of tibia 120. The proximal pin is verified to be substantially vertical to the plane of tibia 120 with the vertical laser beam and then it is seated through tibia 120. The laser insures accuracy of the pin placement, that is, the pin is desirably substantially perpendicular to the horizontal plane of the tibia.

Precision fixation device 10 is substantially preassembled for a left or right leg surgery by an assistant. Assembled device 10 is slid over upper tibial positioning pin 58 via upper tibial positioning aperture 56. The lower tibial positioning pin 64 is positioned in the distal one-third of tibia 120 via lower tibial positioning aperture 62. A 5 mm skin incision makes a window for pin 64 to enter the bone of tibia 120. The laser may now be used to verify that device 10 is in a substantially perfect horizontal plane and parallel to the plane of the tibia 120. Device 10 is generally held tight against the proximal tibia 120 and the lower 64 pin is then set. The laser again is used to verify the absolute vertical position of the lower tibial pin 64 as it is seated through tibia 120. Device 10 may be secured to pins 58 and 64 using set screws (not shown) or other temporary fixation mechanism as known in the art and then the laser may again be used to verify that device 10 is in a substantially perfect horizontal plane and parallel to the plane of the tibia 120. The position of device 10 may be incrementally adjusted until the desired horizontal position is obtained.

The appropriate cutting blade is generally chosen prior to surgery. Any diameter blade may be used in connection with device 10. Saw fixation member 68 is slid onto the first and second slide bars 70 and 72, and saw 82 is placed within saw fixation member 68.

With the position of jig assembly 12 fixed and saw 82 fixed within saw fixation member 68, positioning arm 202 and guide leg 204 are used to accurately position saw 82 in the exact desired position to cut through the tibia 120. Positioning arm 202 can be adjusted by sliding positioning arm relative to screw 104 of adjustment member 96c and further adjusted by rotating positioning arm 202 about screw 104 of adjustment member 96c as shown. The position of positioning arm 202 may be temporarily fixed during the procedure by tightening screw 104 of adjustment member 96c thereby compressing positioning arm 202 against center jig member 16. Further, the angle of incidence between guide leg 204 and positioning arm 202 may be adjusted by loosening screw 104 of adjustment member 96d and rotating guide leg 204 relative to positioning arm 202 and the position of guide leg 204 may be temporarily fixed for the procedure by tightening adjustment member 96d. Positioning arm 202, guide leg 204, and adjustment members 96c and 96d allow a practitioner to adjust the exact location of saw 82 for the cut independently of the location of the pins 58 and 64. This allows for lessening the practitioners reliance on an exact, accurate and precise placement of pins 58, 64 and, in fact, increases the accuracy of the saw cut and reduces the difficulty of the procedure. Once the desired location of the saw is obtained, the blade of saw 82 is positioned substantially perpendicular to the horizontal axis of tibia 120 so that the proximal edge of the saw 82 will cut the proximal end of tibia 120 and set screws (not shown) are tightened within screw apertures 77 to prevent saw 82 from rotating within saw fixation member 68. Positioning arm 202 and guide leg 204 accurately place saw 82 to make a precision perpendicular cut of tibia 120, minimizing bone and/or soft tissue injury.

Prior to commencing operation of the saw, the vertical alignment of saw 82 may be verified using the laser guide or other method. Saw 82 should not move out of the precision plane when it is fixed in position by the saw fixation member 68 on slide bars 70 and 72. Saw 82 is then activated and, with aggressive flushing, tibia 120 is cut approximately two-thirds of the way through. At 90 pounds of pressure, saw 82 is capable of guiding itself through the bone of tibia 120 thereby insuring a substantially perfect 90° cut in all planes relative to tibia 120. The saw blade is then retracted and chisel marks are made to accurately delineate the “mm” of rotation desired. The saw cut is completed with rigorous irrigation by inserting saw 82 into the previous saw cut and activating saw 82. The saw blade is not rotated or twisted by the surgeon, but instead allowed to slowly cut by the actual blade vibration on tibia 120.

Once tibia 120 is cut, jig assembly 12 is checked to confirm that it is secure. The saline soaked gauze is removed from the lateral aspect of tibia 120. A threaded ⅛″ pin is drilled medial to lateral, obliquely, through the proximal cut fragment, close to the saw line. A second pin is then used to rotate the proximal cut segment so that the rotation is complete and the chisel lines meet. A 1/16″ threaded pin is inserted just lateral to the patellar ligament attachment to the tibial crest and is driven posterior into the proximal cut fragment.

Prior to plating, the cut line is visualized for plate placement. The appropriate TPLO plate is contoured to fit the cut surface and proximal shaft of tibia 120. Care is taken to get a perfect anatomical fit. The distal 3 holes are drilled, tapped, and screws placed in a neutral position. Holes 4 and 5 are drilled and 4.0 mm cancellous screws are placed in a loading position, but are tightened together. Hole 6 is drilled parallel to the tibial plateau, and a 4.0 mm cancellous screw is tightened.

A culture is taken prior to closure. 0-PDS is used to close the periosteum and pes ansorinus group over the plate with simple interrupted sutures. The fascia and subcutaneous are closed with 2-0 PDS sutures. If possible, a subcuticular pattern is run with 2-0 vicryl. Stainless steel staples close the skin. A light pressure wrap over the incision and down tibia 120 to the hock joint is applied. Post-op x-rays are taken to evaluate the bone cut line, closure of the saw line, and final degrees of rotation.

From the foregoing, it may be seen that the inventive precision fixation device and method of using the same is particularly well suited for the proposed usages thereof. Furthermore, since certain changes may be made in the above invention without departing from the scope hereof, it is intended that all matter contained in the above description or shown in the accompanying drawing be interpreted as illustrative and not in a limiting sense. It is also to be understood that the following claims are to cover certain generic and specific features described herein.

I claim:

1. A veterinary precision fixation device comprising:
a jig assembly having a center jig member having a first and a second end, a first jig leg pivotally coupled to said first end, and a second jig leg pivotally coupled to said second end;
a saw positioning assembly operably coupled to said center jig member to provide radial and linear translation relative to said jig assembly; and
a saw guide assembly having a saw fixation member slidably coupled to at least one slide bar wherein said slide bar is coupled to said saw positioning assembly.

2. The device of claim 1 wherein a position of said saw guide assembly may be linearly and radially adjusted relative to said jig assembly using said saw positioning assembly.

3. The device of claim 2 wherein said saw positioning assembly includes a positioning arm and a guide leg, said positioning arm having a first end and a second end and being operably coupled to said center jig member, and said guide leg pivotally coupled to said first end of said positioning arm.

4. The device of claim 3 wherein said at least one slide bar is coupled to said guide leg.

5. The device of claim 4 further comprising an adjustment member being configured for selectively tightening said positioning arm to said center jig member.

6. The device of claim 5 further comprising an adjustment member being configured for selectively tightening said guide leg to said positioning arm.

7. The device of claim 6 wherein said saw fixation member is slidably coupled to two slide bars, each of said two slide bars being coupled to said guide leg.

8. The device of claim 1 wherein said first jig leg further comprises an upper tibial positioning aperture configured for receiving an upper tibial positioning pin.

9. The device of claim 1 wherein said second jig leg further comprises a lower tibial positioning aperture configured for receiving a lower tibial positioning pin.

10. The device of claim 1 wherein said saw fixation member defines a saw positioning aperture configured to receive an oscillating saw therein.

11. The device of claim 1 wherein said veterinary fixation device is constructed of a material selected from the group consisting of stainless steel, plastic, and combinations thereof.

12. A method for performing a tibial plateau leveling osteotomy comprising the steps of:
providing a veterinary precision fixation device including a saw guide assembly, a saw positioning assembly, and a jig assembly having a center jig member having a first and a second end, a first jig leg having an upper tibial positioning aperture extending therethrough wherein said first jig leg is pivotally coupled to said first end, and a second jig leg having a lower tibial positioning aperture extending therethrough wherein said second jig leg is pivotally coupled to said second end;
creating a first pilot hole proximate an animal patient's upper tibia;
inserting an upper tibial positioning pin through said upper tibial positioning aperture and into said first pilot hole;
creating a second pilot hole proximate an animal patient's lower tibia;
inserting a lower positioning pin through said lower tibial positioning aperture and into said second pilot hole;
coupling an oscillating saw with said saw guide assembly wherein said saw is substantially perpendicular to said tibia; and
adjusting a position of said saw relative to said jig assembly using said saw positioning assembly.

13. The method of claim 12 further comprising coupling said fixation device to said upper positioning pin and said lower positioning pin.

14. The method of claim 12 wherein said saw positioning assembly comprises a positioning arm and a guide leg.

15. The method of claim 14 said saw guide assembly further comprises a saw fixation member slidably coupled to at least one slide bar wherein said slide bar is coupled to said guide leg of said saw positioning assembly.

16. The method of claim 14 further comprising an adjustment member configured for selectively tightening said positioning arm of said saw positioning assembly to said center jig member.

17. The method of claim 14 further comprising an adjustment member configured for selectively tightening said guide leg to said positioning arm of said saw positioning assembly.

18. The method of claim 12 further comprising at least one adjustment member configured for selectively tightening said first jig leg to said first end and selectively tightening said second jig leg to said second end.

19. A veterinary precision fixation device comprising:
a jig assembly; and
a saw positioning assembly including a positioning arm and a guide leg;
said positioning arm including a first end, a second end, a top surface, a bottom surface, an inner surface and an outer surface wherein said top surface, bottom surface, inner surface and outer surface define a body and wherein said body includes a slot having a length, said slot extending through said body from said top surface to said bottom surface;
said positioning arm including a cutout portion extending inwardly from said first end between said top surface and said bottom surface;
said positioning arm including an aperture extending through said positioning arm from said top surface to said bottom surface and through said cutout portion, said aperture being proximate said first end and wherein said guide leg is pivotally coupled to said positioning arm with an adjustment member that passes through said aperture proximate said first end; and
wherein said positioning arm is slidably and pivotally coupled to said jig assembly.

20. The veterinary precision fixation device of claim 19 further comprising a saw guide assembly coupled to said guide leg wherein said guide assembly comprises at least one guide bar and a saw fixation member wherein said saw fixation member is slidably coupled to said at least one guide bar.
